(19) United States
(12) Patent Application Publication
(10) Pub. No.: US 2011/0224216 A1
(43) Pub. Date: Sep. 15, 2011

(54) Methods of Treating or Preventing Acute Erythema

(75) Inventors: Philippe Andres, Peymeinade (FR); Christian Loesche, Valbonne (FR); Michael Graeber, Lawrenceville, NJ (US)

(73) Assignee: GALDERMA LABORATORIES INC., Fort Worth, TX (US)

(21) Appl. No.: 12/907,543

(22) Filed: Oct. 19, 2010

Related U.S. Application Data
(60) Provisional application No. 61/254,805, filed on Oct. 26, 2009.

(51) Int. Cl. A61K 31/498; A61P 17/00; A61P 29/00

(52) U.S. Cl. 514/249

(57) ABSTRACT

A method of treating or preventing acute erythema in a human in need thereof by topical administration of an effective amount of an alpha adrenergic receptor agonist or pharmaceutically acceptable salt thereof is claimed. The preferred alpha adrenergic receptor agonist is brimonidine. A method of preventing secondary inflammation caused by acute erythema by topical administration of an effective amount of an alpha adrenergic receptor agonist or a pharmaceutically acceptable salt thereof is also claimed.

### CROSS-REFERENCE TO RELATED APPLICATION

This application claims priority from U.S. Provisional Patent Application Ser. No. 61/254,805, filed on Oct. 26, 2009.

### BACKGROUND OF THE INVENTION

Alpha adrenergic receptor agonists such as brimonidine are useful in treating chronic, persistent erythema caused by rosacea, inflammatory skin disorders, telangiectasia, and menopause. See the following U.S. patent and patent applications: U.S. Pat. No. 7,439,241; U.S. Ser. No. 11/137,911; U.S. Ser. No. 12/545,638; U.S. Ser. No. 11/449,079; and U.S. Ser. No. 10/626,037. There remains a need for compositions that prevent and/or treat transient, non-persistent forms of erythema, i.e., acute erythema and that prevents secondary inflammation that results from the acute erythema.

### SUMMARY OF THE INVENTION

In one embodiment, the invention relates to a method of treating acute erythema in a human in need thereof, the method comprising topically administering a pharmaceutically acceptable composition comprising an effective amount of an alpha adrenergic receptor agonist or a pharmaceutically acceptable salt thereof, locally to the site of the acute erythema in the human.

Preferably, the alpha adrenergic receptor agonist is an alpha-1 adrenergic receptor agonist or an alpha-2 adrenergic receptor agonist. More preferably, the alpha adrenergic receptor agonist is a selective alpha-1 adrenergic receptor agonist or a selective alpha-2 adrenergic receptor agonist. Most preferably, the selective alpha-2 adrenergic receptor agonist or a pharmaceutically acceptable salt thereof is brimonidine or brimonidine tartrate. Oxymetazoline is also a preferred alpha adrenergic receptor agonist.

In another embodiment, the invention relates to a method of preventing acute erythema in a human in need thereof, the method comprising topically administering a pharmaceutically acceptable composition comprising an effective amount of an alpha adrenergic receptor agonist or a pharmaceutically acceptable salt thereof, locally to the site of the prospective acute erythema in the human. Preferably, the pharmaceutically acceptable composition comprises an effective amount of brimonidine or a pharmaceutically acceptable salt thereof.

The invention also relates to a method of preventing secondary inflammation in a human in need thereof, the method comprising topically administering a pharmaceutically acceptable composition comprising an effective amount of brimonidine or a pharmaceutically acceptable salt thereof, locally to the site of the prospective secondary inflammation, wherein the secondary inflammation is caused by acute erythema.

### DETAILED DESCRIPTION

The present invention relates to a method of treating acute erythema in a human in need thereof. Acute erythema is defined herein as redness of the skin that appears suddenly as a result of a cause of acute erythema, is non-persistent, and is transient. The redness is non-persistent and transient if it appears as a result of a cause of acute erythema, such as a cause listed below, disappears within a short period of time and does not reappear unless the human is subjected to a second episode of the same cause of acute erythema, or to a different cause. The short period of time during which the acute erythema exists is dependent upon the cause of the acute erythema and can be determined by a person having ordinary skill in the art. The time period may be a few hours, a few days, or possibly a couple of weeks. For example, a mosquito bite may cause acute erythema that lasts for 3 or 4 days.

The non-persistent and transient nature of acute erythema excludes erythema associated with chronic inflammation, such as flushing associated with rosacea or menopause.

There are various causes of acute erythema. Some examples of acute erythema include, but are not limited to, sunburn, cold burns, hot burns, insect bites, physical procedures, and chemical procedures. For example, physical procedures that may induce acute erythema include, but are not limited to, laser rays, ultraviolet light, radio frequency treatment, light-emitting diode treatment, and microderm abrasion treatment. Another example of a physical procedure that may induce acute erythema is radiotherapy for cancer treatment.

Chemical procedures that may induce acute erythema include, but are not limited to, chemical peels, drug treatments on skin, and application of cosmetic products. For example, a drug applied to the skin may lead to irritation manifested by acute erythema. The drug may include an active ingredient that can irritate the skin such as a retinoid.

A cause of acute erythema may also be a combination of any of the above causes that occur simultaneously. For example, a combination of physical and chemical procedures, such as may occur during tanning of the skin and photodynamic therapy, may also induce acute erythema.

The method of treating acute erythema comprises topically administering a pharmaceutically acceptable composition comprising an alpha adrenergic receptor agonist or a pharmaceutically acceptable salt thereof, locally to the site of the acute erythema on the human in an amount sufficient to reduce redness.

Alpha adrenergic receptor agonists are well known in the art. In a preferred embodiment, the alpha adrenergic receptor agonist may be an alpha-1 or alpha-2 adrenergic receptor agonist. The alpha adrenergic receptor agonists included in the invention may or may not show selectivity for either the alpha-1 or alpha-2 adrenergic receptors. For example, some may be considered as being both alpha-1 and alpha-2 adrenergic receptor agonists. More preferably, the alpha adrenergic receptor agonist may be a selective alpha-1 or a selective alpha-2 adrenergic receptor agonist.

Examples of selective alpha-1 adrenergic receptor agonists include oxymetazoline, phenylephrine, and methoxyamine. Examples of selective alpha-2 adrenergic receptor agonists include brimonidine, tetrahydrozaline, naphazoline, xylometazoline, epinephrine, and norepinephrine.

The chemical structures of some selective alpha-1 and selective alpha-2 adrenergic receptor agonists are shown below.

| Chemical Structure | Name |
|---|---|
| 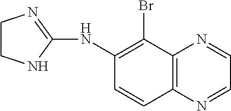 | (5-Bromo-quinoxalin-6-yl)- (4,5-dihydro-1H- imidazol-2-yl)- amine (Brimonidine) |
| 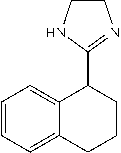 | Tetrahydrozaline |
| 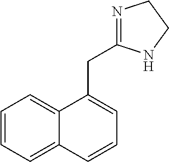 | Naphazoline |
| 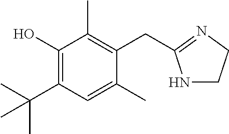 | Oxymetazoline |
| 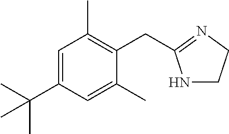 | Xylometazoline |
| 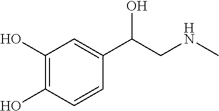 | Epinephrine |
| 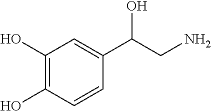 | Norepinephrine |
| 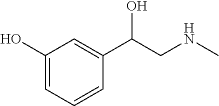 | Phenylephrine |
| 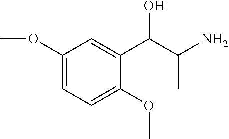 | Methoxyamine |

Brimonidine and its pharmaceutically acceptable salts are preferred embodiments of the invention. Preferably, the active ingredient of the composition is brimonidine tartrate. Oxymetazoline and its pharmaceutically acceptable salts are also preferred embodiments of the invention.

Pharmaceutically acceptable salts for each alpha adrenergic receptor agonists are well known in the art. Pharmaceutically acceptable salt means those salts of compounds of the invention that are safe and effective for topical use in mammals and that possess the desired biological activity. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts. Pharmaceutically acceptable salts are discussed in BERGE ET AL., 66 J. PHARM. SCI. 1-19 (1977), incorporated herein by reference.

Pharmaceutically acceptable compositions include any formulations which are pharmaceutically acceptable for topical delivery of the compounds of the invention. The choice of topical formulation will depend on several factors, including the nature of the symptoms to be treated or prevented, the physiochemical characteristics of the particular compound of the invention and of other excipients present, their stability in the formulation, available manufacturing equipment, and cost constraints.

The pharmaceutically acceptable composition is applied locally to the site of the acute erythema in the human. Acute erythema can occur anywhere on the skin, such as the face, arms, torso, or legs. For example, acute erythema induced by a sunburn may cause redness on the face, shoulders, legs and arms. Therefore, the composition of the invention would be applied to the skin of each of those areas.

To treat acute erythema, the pharmaceutically acceptable compositions of the invention are topically applied directly to the affected area in any conventional manner well known in the art. For example, the compositions are applied by cotton swab or applicator stick, or by simply spreading a formulation of the invention onto the affected area with fingers.

The amount of alpha adrenergic receptor agonist applied to the skin is any amount that is effective in reducing redness due to acute erythema. Generally the minimum amount of an alpha adrenergic receptor agonist in a topical formulation of the invention applied to the affected skin area is about 0.0001 g/cm², preferably about 0.001 g/cm² of skin surface area. The maximum amount of an alpha adrenergic receptor agonist in a topical formulation of the invention applied to the affected skin area is about 0.05 g/cm² to about 0.008 g/cm² of skin surface area. Typically, one to four applications per day are recommended during the term of treatment.

Dosages and dosing frequency will be determined by a trained medical professional depending on the activity of the compound of the invention, the characteristics of the particular topical formulation, the general physical condition of the person being treated, and the severity of the acute erythema treated or prevented.

In general, an alpha adrenergic receptor agonist or pharmaceutically acceptable salt thereof is present in a formulation of the invention in an amount of from about 0.05 percent to about 5 percent of the total weight of the formulation, preferably, of from about 0.07 percent to about 0.7 percent, more preferably, of from about 0.1 percent to about 0.6 percent of the total weight of the formulation.

In one embodiment, the compounds of the invention are delivered to the affected area of the skin in a pharmaceutically acceptable topical carrier. As used herein, a pharmaceutically acceptable topical carrier is any pharmaceutically acceptable formulation that can be applied to the skin surface for topical or dermal delivery of a pharmaceutical or medicament. The combination of a pharmaceutically acceptable topical carrier and a compound of the invention is termed a topical formulation of the invention. Topical formulations of the invention are prepared by mixing a compound of the invention with a topical carrier according to well-known methods in the art, for example, methods provided by standard reference texts such as, REMINGTON: THE SCIENCE AND PRACTICE OF PHARMACY 1577-1591, 1672-1673, 866-885(Alfonso R. Gennaro ed. 19th ed. 1995); Ghosh, T. K.; et al. TRANSDERMAL AND TOPICAL DRUG DELIVERY SYSTEMS (1997), both of which are hereby incorporated herein by reference. The discussion of topical formulations containing alpha adrenergic receptor agonists from U.S. Pat. No. 7,439,241 is incorporated herein by reference.

The topical carriers useful for topical delivery of compounds of the invention can be any carrier known in the art for topically administering pharmaceuticals, for example, but not limited to, pharmaceutically acceptable solvents, such as a polyalcohol or water; emulsions (either oil-in-water or water-in-oil emulsions), such as creams or lotions; micro emulsions; gels; ointments; liposomes; powders; and aqueous solutions or suspensions. The preferred carriers are gels and creams.

In a preferred embodiment, the pharmaceutically acceptable composition contains only one active ingredient, i.e., an effective amount of one alpha adrenergic receptor agonist or a pharmaceutically acceptable salt thereof. In another preferred embodiment, the pharmaceutically acceptable composition may contain more than one active ingredient including an effective amount of more than one alpha adrenergic receptor agonist or a pharmaceutically acceptable salt thereof, or one alpha adrenergic receptor agonist or a pharmaceutically acceptable salt thereof and another pharmaceutically active ingredient.

Other pharmaceutically active ingredients or their pharmaceutically acceptable salts, that may be present in the topical formulations of the invention can include, for example, topical corticosteroids and other anti-inflammatory agents, such as betamethasone, diflorasone, amcinonide, fluocinolone, mometasone, hydrocortisone, prednisone, and triamcinolone; local anesthetics and analgesics, such as camphor, menthol, lidocaine, and dibucaine, and pramoxine; antifungals, such as ciclopirox, chloroxylenol, triacetin, sulconazole, nystatin, undecylenic acid, tolnaftate, miconizole, clotrimazole, oxiconazole, griseofulvin, econazole, ketoconozole, and amphotericin B; antibiotics and anti-infectives, such as mupirocin, erythromycin, clindamycin, gentamicin, polymyxin, bacitracin, and silver sulfadiazine; and antiseptics, such as iodine, povidine-iodine, benzalkonium chloride, benzoic acid, chlorhexidine, nitrofurazine, benzoyl peroxide, hydrogen peroxide, hexachlorophene, phenol, resorcinol, and cetylpyridinium chloride.

The formulations of the invention can be used in combination with other treatments and medications to provide more effective treatment or prevention of acute erythema and symptoms associated therewith. In a preferred embodiment, the topical formulations of the invention are used in combination with treatment regimens and medications well known for treatment of dermatologic disorders, such as those disclosed in The Merck Manual 811-830 (Keryn A. G. Lane et al. eds. 17^{th} ed. 2001), hereby incorporated herein by reference.

Another aspect of the invention relates to a method of preventing acute erythema in a human in need thereof by topically administering a pharmaceutically acceptable composition comprising an effective amount of an alpha adrenergic receptor agonist or a pharmaceutically acceptable salt thereof, locally to the site of the prospective acute erythema. The acute erythema may be induced by any of the causes described above, such as by exposure to sunburn, cold burns, hot burns, insect bites, physical procedures, chemical procedures, or combinations thereof.

The site of the prospective acute erythema varies depending upon what induces the acute erythema. For example, a person who will be outdoors on a sunny day, may apply the composition to exposed areas of the body, such as the face, shoulders, arms, and legs. In another example, a person susceptible to mosquito bites may apply the composition to their face, legs and arms before going outdoors in the evening.

The pharmaceutically acceptable composition may be applied to the site of the prospective acute erythema at any appropriate period of time prior to, concurrently with, or after the inducement. For example, the pharmaceutically acceptable composition may be applied to a patient's face one or more times during the days or hours prior to the patient undergoing a microderm abrasion procedure, radio frequency treatment, light-emitting diode treatment, etc. Administration of the composition will help in preventing the acute erythema.

Another aspect of the invention relates to a method of preventing secondary inflammation in a human in need thereof, the method comprising topically administering a pharmaceutically acceptable composition comprising an effective amount of brimonidine or a pharmaceutically acceptable salt thereof, locally to the site of the prospective secondary inflammation.

Secondary inflammation is defined as an inflammation caused by acute erythema. For example, physical and chemical procedures that lead to acute erythema may also cause tissue damage and trigger inflammation, especially if untreated. The site of the prospective secondary inflammation is the place where acute erythema is or was present. Administration of the composition will help in preventing the secondary inflammation.

### EXAMPLES

### Example 1

### Synthesis of Brimonidine (5-Bromo quinoxalin-6-yl)-(4,5-dihydro-1H-imidazol-2-yl)-amine

To a stirred solution of 6-amino-5-bromoquinoxaline hydrobromide (10 g) in distilled water (150 ml) is added thiophosgene (3 ml). The solution is stirred for two hours at room temperature and the resultant precipitate is collected by filtration, washed with water, and dried to afford 5-bromo-6-isothiocyanato-quinoxaline.

The 5-bromo-6-isothiocyanato-quinoxaline (3.5 g) is directly dissolved in benzene (400 ml) and added dropwise to a well-stirred solution of ethylene diamine (15 g.) in benzene (50 ml). During a period of about two hours, an oil separates as a lower layer. The upper benzene layer is poured off and the oil is washed with diethyl ether and then dissolved in methanol (500 ml). The methanolic solution is refluxed until hydrogen sulfide evolution ceases. The methanolic solution is concentrated in vacuo to a volume of approximately 100 ml upon which a yellow solid precipitates. The precipitate is collected by filtration and recrystallized from methanol to afford of (5-Bromo-quinoxalin-6-yl)-(4,5-dihydro-1H-imidazol-2-yl)-amine: m.p. 250-251° C.

### Example 2

### Synthesis of Brimonidine Tartrate 5-bromo-6-(2-imidazolidinylideneamino) quinoxaline L-tartrate

The tartrate salt of brimonidine can be synthesized by adding (L)-(+)-tartaric acid to a solution of brimonidine in aqueous methanol. The brimonidine tartrate will separate out of solution.

### Example 3

Gel Formulation

| Ingredient | Weight Percent |
|---|---|
| Brimonidine tartrate | 0.18% |
| Carbomer 934P | 1.25% |
| Methylparaben | 0.3% |
| Phenoxyethanol | 0.4% |
| Glycerin | 5.5% |
| 10% Titanium dioxide | 0.625% |
| Propylene glycol | 5.5% |
| 10% NaOH Solution | 6.5% |
| DI Water | QS |
| TOTAL | 100% |

### Example 4

Cream Formulation

| Ingredient | Weight Percent |
|---|---|
| Brimonidine tartrate | 0.18% |
| Phenoxyethanol | 0.8% |
| Methylparaben | 0.2% |
| Propylparaben | 0.05% |
| Disodium EDTA | 0.01% |
| Butylated Hydroxytoluene | 0.05% |
| PEG-300 | 4.0% |
| PEG-6 Stearate (and) | 7.5% |
| Glycol Stearate (and) PEG- | |
| 32 Stearate | |
| Cetostearyl alcohol | 4.0% |
| Caprylic capric | 7.0% |
| triglycerides | |
| Diisopropyl adipate | 7.0% |
| Oleyl alcohol | 7.0% |
| Lanolin USP | 2.0% |
| Ceteareth-6 (and) Stearyl | 2.0% |
| Alcohol | |
| Ceteareth-25 | 2.0% |
| Tartaric Acid | 0.001% |
| DI Water | 56.209% |
| TOTAL | 100% |

We claim:

1. A method of treating acute erythema in a human in need thereof, the method comprising topically administering a pharmaceutically acceptable composition comprising an effective amount of an alpha adrenergic receptor agonist or a pharmaceutically acceptable salt thereof, locally to the site of the acute erythema in the human.

2. The method of claim 1, wherein the acute erythema is a skin condition that appears suddenly, is non-persistent, and is manifested by transient redness of the skin.

3. The method of claim 2, wherein the acute erythema is induced by sunburn, cold burns, hot burns, insect bites, physical procedures, chemical procedures, or combinations thereof.

4. The method of claim 3, wherein the acute erythema is induced by physical procedures selected from the group consisting of laser rays, ultraviolet light, radio frequency, radiotherapy, light-emitting diode, and microdermabrasion treatments.

5. The method of claim 3, wherein the acute erythema is induced by chemical procedures selected from the group consisting of chemical peelings, drug treatments on skin, and application of cosmetic products.

6. The method of claim 5, wherein the chemical procedure comprises application of a retinoid.

7. The method of claim 3, wherein the acute erythema is induced by physical and chemical procedures selected from the group consisting of tanning, photodynamic therapy, and combinations thereof.

8. The method of claim 1, wherein the alpha adrenergic receptor agonist is an alpha-1 adrenergic receptor agonist or a pharmaceutically acceptable salt thereof.

9. The method of claim 8, wherein the alpha adrenergic receptor agonist is a selective alpha-1 adrenergic receptor agonist or a pharmaceutically acceptable salt thereof.

10. The method of claim 9, wherein the selective alpha-1 adrenergic receptor agonist is selected from the group consisting of oxymetazoline, phenylephrine, methoxyamine, and pharmaceutically acceptable salts thereof.

11. The method of claim 10, wherein the selective alpha-1 adrenergic receptor agonist is oxymetazoline.

12. The method of claim 1, wherein the alpha adrenergic receptor agonist is an alpha-2 adrenergic receptor agonist or a pharmaceutically acceptable salt thereof.

13. The method of claim 12, wherein the alpha adrenergic receptor agonist is a selective alpha-2 adrenergic receptor agonist or a pharmaceutically acceptable salt thereof.

14. The method of claim 13, wherein the selective alpha-2 adrenergic receptor agonist is selected from the group consisting of brimonidine, tetrahydrozaline, naphazoline, xylometazoline, epinephrine, norepinephrine, and pharmaceutically acceptable salts thereof.

15. The method of claim 14, wherein the selective alpha-2 adrenergic receptor agonist is brimonidine or a pharmaceutically acceptable salt thereof.

16. The method of claim 15, wherein the selective alpha-2 adrenergic receptor agonist or a pharmaceutically acceptable salt thereof is brimonidine tartrate.

17. The method of claim 15, wherein the weight percentage of brimonidine in the composition is at least 0.05% and at most about 5%.

18. The method of claim 17, wherein the weight percentage of brimonidine in the composition is at least 0.07% and at most 0.7%.

19. The method of claim 18, wherein the weight percentage of brimonidine in the composition is at least 0.1% and at most 0.6%.

20. The method of claim 1, wherein the site of the acute erythema is the face, arms, torso, or legs.

21. The method of claim 1, wherein the composition comprises one active agent consisting of an alpha adrenergic receptor agonist or a pharmaceutically acceptable salt thereof.

22. A method of treating acute erythema in a human in need thereof, the method comprising topically administering a pharmaceutically acceptable composition comprising an effective amount of brimonidine or a pharmaceutically acceptable salt thereof, locally to the site of the acute erythema in the human.

23. A method of preventing acute erythema in a human in need thereof, the method comprising topically administering a pharmaceutically acceptable composition comprising an effective amount of an alpha adrenergic receptor agonist or a pharmaceutically acceptable salt thereof, locally to the site of the prospective acute erythema in the human.

24. The method of claim 23, wherein the prospective acute erythema is induced by exposure to sunburn, cold burns, hot burns, insect bites, physical procedures, chemical procedures, or combinations thereof.

25. The method of claim 24, wherein the pharmaceutically acceptable composition is applied prior to or concurrently with receiving a sunburn, cold burn, hot burn, or insect, or undergoing a physical procedure or chemical procedures; or combinations thereof.

26. A method of preventing acute erythema in a human in need thereof, the method comprising topically administering a pharmaceutically acceptable composition comprising an effective amount of brimonidine or a pharmaceutically acceptable salt thereof, locally to the prospective site of the acute erythema in the human.

27. The method of claim 26, wherein the prospective acute erythema is induced by exposure to sunburn, cold burns, hot burns, insect bites, physical procedures, chemical procedures, or combinations thereof.

28. The method of claim 27, wherein the pharmaceutically acceptable composition is applied prior to or concurrently with receiving a sunburn, cold burn, hot burn, or insect, or undergoing a physical procedure or chemical procedures; or combinations thereof.

29. A method of preventing a secondary inflammation in a human in need thereof, the method comprising topically administering a pharmaceutically acceptable composition comprising an effective amount of brimonidine or a pharmaceutically acceptable salt thereof, locally to the site of the prospective secondary inflammation, wherein the secondary inflammation is caused by acute erythema.
